(19) United States
(12) Patent Application Publication
(10) Pub. No.: US 2009/0209488 A1
(43) Pub. Date: Aug. 20, 2009

(54) Compositions for the treatment of exercise induced asthma

(76) Inventor: Roger Wayne BROWN, Phoenix, AZ (US)

Correspondence Address: Roger W. Brown, Ph.D., 810 W. Villa Maria Dr., Phoenix, AZ, 85023, US

(21) Appl. No.: 12/218,971

(22) Filed: Jul. 21, 2008

Related U.S. Application Data
(63) Continuation-in-part of application No. 12/070,313, filed on Feb. 19, 2008.

(51) Int. Cl. A61K 31/7008; A61P 11/06

(52) U.S. Cl. 514/62

(57) ABSTRACT

In accordance with the present invention, there is to provide a dietary supplement that provides a mammal with the essential carbohydrates needed to maintain proper health and functionality, to fend off illness, to provide an alternative treatment for exercise induced asthma and asthma related illnesses, to lessen the aging process of cells and to provide pets with another level of medications equal to that for humans.

### RELATED APPLICATIONS

This application claims priority on patent application Ser. No. 12/151,394 filed with the PTO on May 7, 2008, patent application Ser. No. 12/079,907 filed with the US PTO on Mar. 31, 2008 and on patent application Ser. No. 12/070,313 filed Feb. 19, 2008, and is a continuation-in-part of patent application Ser. No. 12/070,313 filed Feb. 19, 2008, the entire disclosure of which is incorporated herein.

The present application is related to U.S. Pat. No. 7,276,529, issued Oct. 2, 2007, included by reference herein.

The present application is related to U.S. Pat. No. 6,555,576, issued Apr. 29, 2003, included by reference herein.

The present application is related to U.S. Pat. No. 7,202,220 B2, issued Apr. 10, 2007, included by reference herein.

The present application is related to U.S. Pat. No. 7,199,104 B2, issued Apr. 3, 2007, included by reference herein.

The present application is related to U.S. Pat. No. 7,196,064 B2, issued Mar. 27, 2007, included by reference herein.

The present application is related to U.S. Pat. No. 7,157,431 B2, issued Jan. 2, 2007, included by reference herein.

The present application is related to U.S. Pat. No. 6,929,807 B1, issued Aug. 16, 2005, included by reference herein.

The present application is related to U.S. Pat. No. 3,890,438 A, issued Jun. 1, 1975, included by reference herein.

The present application is related to U.S. Pat. No. 3,947,601 A, issued Mar. 1, 1976, included by reference herein.

The present application is related to U.S. Pat. No. 4,260,603 A, issued Apr. 1, 1981, included by reference herein.

The present application is related to U.S. Pat. No. 4,466,958 A, issued Aug. 1, 1984, included by reference herein.

The present application is related to U.S. Pat. No. 4,777,045 A, issued Oct. 1, 1988, included by reference herein.

The present application is related to U.S. Pat. No. 4,871,557 A, issued Oct. 1, 1989, included by reference herein.

The present application is related to U.S. Pat. No. 5,612,039 A, issued Mar. 1, 1997, included by reference herein.

The present application is related to U.S. Pat. No. 5,827,526, issued Oct. 1, 1998, included by reference herein.

The present application is related to U.S. Pat. No. 7,244,706, issued Jul. 17, 2007, included by reference herein.

The present application is related to U.S. Pat. No. 7,323,179, issued Jan. 29, 2008, included by reference herein.

US Class: 514/8; 514/23; 514/54; 514/62; 424/725

### FIELD OF THE INVENTION

The present invention relates to the field of dietary supplements promoting good nutritional health and, more particularly, to the compositions of carbohydrates as dietary supplements that are required by mammals for good health and specifically to provide an alternative treatment for exercise induced asthma and asthma related illnesses.

### BACKGROUND OF THE INVENTION

Asthma is a pulmonary disease characterized by reversible airway obstruction, airway inflammation, and increased airway responsiveness to a variety of stimuli. Exercise induced asthma (EIA) is a condition of respiratory difficulty that is triggered by aerobic exercise lasting several minutes. Patients usually complain of exercise related respiratory symptoms. This complaint is much more common among children and younger athletes but can be seen at any age. Symptoms during or following exercise include chest tightness, chest pain, cough, shortness of breath, wheezing, stomach ache and fatigue. http://kidshealth.org/parent/medical/asthma/exercise_asthma.html

EIA affects 12 to 15% of the population in the United States. EIA is experienced by 90% of asthmatic individuals and 35 to 45% of people with allergic rhinitis. Even when eliminating those with rhinitis and allergic asthma, a 3 to 10% incidence of EIA is seen in the general population. EIA seems to be somewhat more prevalent in some winter or cold weather sports then in hot weather sports. Some studies have demonstrated rates as high as 35% or even 50% in competitive caliber figure skaters, ice hockey players, and cross country skiers. http://www.emedicine.com/sports/topic155.htm

Although the exact mechanism is unknown, there are two predominant theories as to how the symptom of EIA is triggered. One is hyperosmolarity theory or airway humidity theory, which suggests that air movement through the airway results in relative drying of the airway. This in turn is believed to trigger a cascade of events that results in airway edema, secondary to hyperemia and increased perfusions in an attempt to combat the drying. This is believed to lead to the release of mediators that cause bronchoconstriction. The mediators include histamine, prostaglandin, and leukotriene.

The other theory is airway rewarming theory. It is based on airway cooling and assumes that the air movement in the bronchial tree results in a decreased temperature of the bronchi, which may also trigger a hyperemic response in an effort to heat the airway. This leads to congested vessels, fluid exudation from the blood vessels into the submucosa of the airway wall, and mediator release with subsequent bronchoconstriction.

Causes of EIA can be divided into the categories of medical, environmental, and drug related. Poorly controlled asthma or allergic rhinitis results in increased symptoms with exercise. Secretions of hay fever can aggravate EIA. Viral, bacterial, and other forms of upper respiratory infections also aggravate the symptoms of EIA. Excess of pollens or molds in the air can exacerbate EIA. Pollutants such as cigarette smoke, sulfur dioxide and nitrogen oxide in the air are irritants to the airways and can lower the threshold for symptomatic bronchospasm. Chemicals used in certain sports for environmental maintenance can worsen EIA symptoms. Aspirin and beta blockers are also known as asthmatogenic agents.

Anyone who has asthma is at increased risk of getting exercise induced asthma. A number of additional factors may increase the chances of developing asthma in general. These factors include:
Having one or both parents with asthma
Living in a large urban area, especially the inner city, which may increase exposure to environmental pollutants
Exposure to secondhand smoke
Exposure to occupational triggers, such as chemicals used in farming and hairdressing, and in paint, steel, plastics and electronics manufacturing
Respiratory infections in childhood
Low birth weight
Obesity
Gastroesophageal reflux disease (GERD)

Because exercise induced asthma has the same symptoms and results from the same airway reaction involved in regular asthma, standard asthma medications can control it. The only difference between these two diseases, Asthma and EIA, is the trigger mechanism. Because of this the method of treatment and the medications used for either of these two diseases are effective against the other disease as well. Depending on the severity and frequency of the symptoms, a doctor may prescribe one, or more, of the following treatments (http://www.nhlbi.nih.gov/health/dci/Diseases/Asthma/Asthma_Treatments.html):

A short acting inhaled bronchodilator, such as albuterol (Proventil, Ventolin, ProAir) or pirbuterol (Maxair), is used 15 minutes before exercise to prevent symptoms for about four hours. A mast cell stabilizer, such as cromolyn sodium (Intal) or nedocromil sodium (Tilade), is used 15 to 60 minutes before exercise to prevent symptoms for about four hours.

A long acting beta-2 agonist (LABA), such as salmeterol (Serevent Diskus) and formoterol (Foradil), taken 30 minutes before exercise can relieve symptoms for up to 12 hours. The Food and Drug Administration (FDA) issued a public health advisory stating that certain LABAs may increase the risk of a severe asthma attack in some people. LABAs are used in combination with inhaled corticosteroids.

A leukotriene modifier, such as montelukast sodium (Singulair) or zafirlukast (Accolate), can produce beneficial effects lasting up to 24 hours. These drugs are helpful in reducing airway inflammation when exercise induced symptoms are a feature of chronic asthma, so are taken every day.

A corticosteroid inhaler, such as fluticasone (Flovent) mometasone (Asmanex), budesonide (Pulmicort) or triamcinolone (Azmacort). Your doctor may recommend that you use a corticosteroid inhaler every day if you wheeze whenever you exert yourself or if allergies and irritants also trigger your symptoms. These drugs reduce inflammation. In addition, you may need daily doses of a long acting bronchodilator.

Controlling asthma can also help to avoid serious side effects from long term use of some medications used to stabilize severe asthma. Using inhaled corticosteroids, which have fewer side effects than oral corticosteroids, can help reduce the need for emergency treatment of asthma.

Traditionally, patients have been advised to take corticosteroids on a daily basis. However, a 2005 study suggested that intermittent corticosteroid therapy may be appropriate for some patients with mild persistent asthma. In the Improving Asthma Control Trial (IMPACT), researchers found that patients with mild persistent asthma who used an inhaled corticosteroid (budesonide) on an as needed basis to control acute symptoms had similar lung function and quality of life outcomes as patients who used the drug daily. The researchers emphasize that patients with severe asthma should adhere to a daily dosage schedule, and that all patients with asthma should consult with their doctor to discuss any changes in medication regimen. Optimal timing of the dose is important and may vary depending on the medication. Most of the newer inhaled steroids and even some older ones are now available as a single daily dose.

Inhaled steroids are generally considered safe and effective and only rarely cause any of the more serious side effects reported with prolonged use of oral steroids. Side effects of inhaled steroids are the following:
The most common side effects are throat irritation, hoarseness, and dry mouth. These effects can be minimized or prevented by using a spacer device and rinsing the mouth after each treatment.
Rashes, wheezing, facial swelling (edema), fungal infections (thrush) in the mouth and throat, and bruising are also possible but not common with inhalators.
A 2001 study reported a higher risk for cataracts in patients over age 40. No higher risk was observed in younger people. Some studies report a higher risk for bone loss in patients who take inhaled steroids regularly, a side effect which is known to occur with oral steroids. A number of bone preserving medications are now available that might safely offset this effect. There is some concern that the more potent drugs, particularly fluticasone, suppress the adrenal system, which secretes natural steroids, to a greater degree than other steroid inhalants. This is a serious side effect of oral steroids.

Long acting beta2-agonists are used in combination with inhaled corticosteroids for treating patients with moderate to severe asthma. These drugs include include salmeterol (Serevent Diskus) and formoterol (Foradil Aerolizer). Single inhalers that combine salmeterol and the corticosteroid fluticasone (Advair Diskus) and formoterol and the corticosteroid budesonide (Symbicort) are also available.

Long acting beta2-agonists are used for preventing an asthma attack (not for treating attack symptoms). The effects of one dose of a long acting beta2-agonist last for about 12 hours, so they are particularly effective during the night. These drugs also may be used for prevention of exercise induced asthma in people and to protect against aspirin induced asthma.

However, research indicates that long acting beta2-agonists can worsen asthma by increasing symptom severity. These drugs may also increase the risk for asthma related deaths. Experts are still trying to determine when long acting beta2-agonists should be added to an asthma treatment plan.

The side effects of long acting beta2-agonists are similar to the short acting drugs. In 2003, a “black box” warning was added to product packaging for drugs that contain salmeterol, including Serevent Diskus, and Advair Diskus. The warning was based on a study that demonstrated more serious and even fatal asthma episodes in patients who used the drug than in patients who used a placebo. The risk for serious asthma episodes with salmeterol appears to be highest in African Americans and elderly patients with severe asthma.

In 2006, the FDA updated the warning to include formoterol (Foradil Aerolizer). Warnings for salmeterol and formoterol products emphasize that these medicines can increase the risk of severe asthma episodes. If these episodes occur, they can be fatal. Long acting beta2-agonists require up to 20 minutes to achieve effectiveness, and there is a danger of overdose if a patient is not aware of this delay and takes additional doses to achieve faster relief. The FDA recommends that patients:
Use long acting beta2-agonists only if other medicines (such as steroids) have not helped control asthma
Use a short acting bronchodilator, not a long acting beta2-agonist, to treat sudden wheezing
Do not use long acting beta2-agonists to treat wheezing that is getting worse. Call your doctor if this situation occurs
Do not stop using any asthma medicines without first talking to your doctor

Leukotriene antagonists (also called anti-leukotrienes or leukotriene modifiers) are oral medications that block leukotrienes. Leukotrienes are powerful immune system factors that, in excess, produce a battery of damaging chemicals that can cause inflammation and spasms in the airways of people with asthma. As with other anti-inflammatory drugs, leukotrienes are used for prevention and not for treating acute asthma attacks.

Leukotriene antagonists include zafirlukast (Accolate), montelukast (Singulair), zileuton (Ziflo), and pranlukast (Ultair, Onon). These drugs are proving to be effective for long term prevention of asthma, including exercise induced asthma and aspirin (or NSAID) induced asthma. Most studies to date still report better success with inhaled corticosteroids than with the leukotriene antagonists. Their anti-inflammatory actions are different from those of steroids, however, and combinations of the two drugs are being tried. A 2002 analysis of 13 studies, however, reported only modest benefits when anti-leukotrienes were added to corticosteroids. The combination did improve asthma control in some of the studies, but they did not reduce corticosteroid use. (In all but one of these studies the subjects were adults.) Gastrointestinal distress is the most common side effect of leukotriene antagonists. Very few other side effects have been reported.

Of some concern are reports of Churg Strauss syndrome in a few people taking zafirlukast or montelukast. Churg Strauss syndrome is very rare, but it causes blood vessel inflammation in the lungs and can be life threatening. Oral steroids quickly resolve the problem. Usually the syndrome has occurred in patients who were tapering off steroids and changing over to the leukotrienes antagonists. Some experts believe that, in such cases, the steroids may simply have masked the presence of the disorder, which then developed when the steroid drugs were withdrawn. Symptoms include severe sinusitis, flu like symptoms, rash, and numbness in the hands and feet.

Other concerns are indications of liver injury in patients taking zileuton and zafirlukast when taken at higher than standard doses. No adverse effects on the liver have been reported to date with montelukast.

Theophylline relaxes the muscles around the bronchioles and also stimulates breathing. One study reported that it may also have anti-inflammatory qualities even in low doses. Available in tablet, liquid, and injectable forms, some theophylline sustained release tablets and capsules have a long duration of action and can, therefore, be taken once or twice a day with good results.

If theophylline is not taken exactly as prescribed, an overdose can easily occur. Toxicity can cause nausea, vomiting, headache, insomnia, and, in rare cases, disturbances in heart rhythm and convulsions.

The risks for these adverse effects are small if the drug is taken exactly as prescribed, but the following precautions should be noted:
Chronic smokers metabolize theophylline much more quickly and require higher doses of the drug than nonsmokers; prolonged release versions are helpful for such people.
Too much caffeine can increase the concentration of this drug and the amount of time it stays in the body.
Theophylline also interacts with many other drugs that are taken for other common medical conditions, including asthma. Exercise caution when using beta2-agonists and theophylline together.
No one with a peptic ulcer should take theophylline. The elderly and anyone with heart disease, liver disease, hypertension, seizure disorders, or congestive heart failure, should take theophylline with caution. Of special note, people with heart conditions who take theophylline orally face an increased risk for sudden death from heart related causes.

Omalizumab (Xolair) is FDA approved for patients age 12 and older who have moderate to severe persistent asthma related to allergies. The first drug of this type to be approved for asthma, omalizumab is a monoclonal antibody (MAb), a genetically developed drug designed to attack very specific targets. Omalizumab is administered by injection every 2 to 4 weeks. It is used only to treat patients whose symptoms are not controlled by inhaled corticosteroids.

Omalizumab prevents the antibody Immunoglobulin E (IgE) from triggering the inflammatory events that lead to asthmatic attacks. Studies have shown excellent benefits of the drug, including a reduced need for corticosteroids, fewer hospitalizations, and significant symptomatic improvements.

However, about 1 in 1,000 patients who take omalizumab develop anaphylaxis (a life threatening allergic reaction). In 2007 the FDA requested the manufacturers of omalizumab to put a “boxed warning” on the medicine's label emphasizing the drug's risk for anaphylaxis. The boxed warning notes that patients can develop anaphylaxis after any dose of omalizumab, even if they had no reaction to a first dose. Anaphylaxis may occur up to 24 hours after the dose is given.

The FDA recommends that health care providers observe patients for at least 2 hours after an injection. Patients should also carry emergency self-treatment for anaphylaxis (such as an Epi-Pen) and know how to administer it. With an Epi-Pen, or similar auto-injector device, patients can quickly give themselves a life saving dose of epinephrine.

Anaphylaxis symptoms include:
Difficulty breathing
Chest tightness
Dizziness
Fainting
Itching and hives
Swelling of the mouth and throat

Even though much is being done to safeguard the patient there is still a significant need for a safer and more effective method of treating, preventing and managing exercise induced asthma, particularly for patients that are refractory to conventional treatments, while reducing or avoiding the toxicity and/or side effects associated with conventional therapies.

Mammal's bodies produce a large number of different types of chemicals that the body uses to ward off disease, retard cell degradation, maintain memory and maintain overall body health. These chemicals are produced as a byproduct of what the mammal has eaten. If all of the right foods are eaten in the proper amounts then the body will produce enough of all of the chemicals required to keep it functioning properly. Over the years people have sought after which chemicals are actually necessary for good health and which ones are just good. As this field is evolving more and more information is being discovered about what chemicals mammal's bodies require for proper functionality.

Over the last ten years a lot of research has been done concerning cell communications and its importance to a properly functioning mammal's body. This research indicates that there are eight essential sugars that all mammals need in order to stay healthy; http://www.glyconutrients-center.org/ and http://www.glyconutrientsreference.com/. Six of these carbohydrates (sugars) are generally missing in the diets of most humans and seven are missing from the diet of animals. However, very small concentrations of these missing carbohydrates are contained in various plants and some sea products. A synopsis of these seven essential sugars/carbohydrates and what functions they have been found to influence follows:

D-Galactose is readily available in human diets but not in animal diets. It is obtained from the conversion of lactose (milk sugar) and is also easily obtained from dairy products UNLESS you suffer from lactose intolerance or are a vegetarian who does not eat dairy products.

D-Mannose is not readily available in our diets. The most popular source is Aloe Vera. It is also available in tiny quantities in the bran of whole wheat. However, it is very unstable and must be taken fresh from the plant and properly standardized to be of any benefit. It plays a profound role in cellular interactions and has even been known to lower blood sugar levels. It is absolutely vital to proper immune defenses against microbial invaders and has a natural and powerful anti-inflammatory effect. This sugar is readily available in supplemental form. Good for: Wound healing, Diabetics, Anti-viral, Anti-inflammatory and Arthritis.

N-Acetyl-Glucosamine is not readily available in our diets. It is particularly beneficial for cartilage regeneration and joint inflammation. Glucosamine derivatives are well-known natural medicine for arthritic conditions comes from this sugar compound. It has many more therapeutic effects and deficiencies or malfunctions of this sugar have been linked to diseases of the bowel. Derivatives of this sugar are readily available in supplemental form. Good for: Wound repair, Range of motion, Insulin production, Arthritic conditions, Learning, HIV and Vision.

L-Fucose is not readily available in our diets but is readily found in breast milk, astragalus herb, in several medicinal mushrooms, and in certain brown algae. It has numerous well-documented benefits for the immune system and has been shown to inhibit some cancer growth and metastasis. Good for: Long term memory, Cancer and tumors and Skin allergies.

D-Xylose is not readily available in our diets. It is often seen in sugarless gums, candies, etc. in that it has a sweet taste but does not cause tooth decay. It has recently been added to nasal sprays and appears to discourage the binding of allergens and pathogens to mucous membranes. It also has known anti-bacterial and fungal properties and may help prevent certain cancers. Good for: Anti-fungal and gram negative bacteria.

N-Acetyl-Neuraminic Acid is not readily available in our diets but is another sugar that abounds in breast milk and dramatically impacts brain function and growth. It, too, boosts immune function and has documented anti-viral actions. Interestingly, in certain disease states, the ability to digest this sugar is impaired. Good for: 1000× Best Anti-viral known, Kidney stones, Asthma, Learning and Arthritis.

N-Acetyl-Galactosamine is not readily available in our diets. It is the least known of the essential sugars although it appears to inhibit the growth of some tumors and, like the other sugars, plays an individual role in keeping cellular messages clear and promptly delivered. Most of these sugars do not involve or require insulin for their use and go directly to the cells where they are incorporated into the cell structure wherever they are needed. Good for: Heart disease, Aging (cell rejuvenation), Joint functioning and Vision.

The actual body requirements for these missing carbohydrates has been hard to estimate because of their rarity and because of this the FDA has not set a lower daily intake limit on any of them. However, research has indicated a level of dose for each of these carbohydrates required to produce noticeable effects. The base level for each of these carbohydrates is about 0.005 mg/kg of body weight or 0.4 mg/day for a 150 pound mammal. These levels correspond to base levels of other medications.

The base level is not the required level to start seeing results but the level below which nothing much has been seen. The general therapeutic levels are above 0.1 mg/kg of body weight or 8 mg for a 150 pound mammal. These findings indicate that the minimum required level for these essential carbohydrates is a hundred times larger than is available in natural foods including the specially prepared supplements designed to alleviate the missing carbohydrate deficiency.

The seven essential sugars/carbohydrates are: D-Galactose, L-Fucose, D-Mannose, D-Xylose, N-Acetyl-Glucosamine, N-Acetyl-Neuraminic Acid, and N-Acetyl-Galactosamine. Of these seven carbohydrates D-Mannose and Glucosamine derivatives are available as full strength supplements from a large number of over the counter drug stores and D-Galactose is available from specialty suppliers. The remaining four carbohydrates are much too expensive for companies to currently package in full strength so all that is generally available to the public are very low concentration food substitutes.

While there are several different companies selling glyconutrients only one of them has filed for patent protection in the US. This company is Mannatech, Inc. of Coppell Tex. and they sell their glyconutrients through a chain of 500,000 independent dealers worldwide. They filed an initial U.S. patent application in 1997 and was granted a U.S. patent on Aug. 16, 2005; U.S. Pat. No. 6,929,807 B1. Since then they have filed four additional amendments to this one patent. They were granted additional patent numbers which are: U.S. Pat. No. 7,157,431 B2, U.S. Pat. No. 7,196,064 B2, U.S. Pat. No. 7,199,104 B2, and U.S. Pat. No. 7,202,220 B2.

Their first patent sets out eight essential sugars and ties these sugars to food sources where they can be found. However, they don't disclose the actual amount of the various essential sugars/carbohydrates in these food sources. Their web site is located at: https://www.mannatech.com/Default.aspx

They produce specific supplements used to address several different conditions that arise in mammals: weight management, alcoholism, nutrition, wellness management, lifestyle management, growth essentials, performance management, skin care and performance nutrition. All of these products are different mixes of the same basic foods, as seen above, and as can be seen they contain very little of what is required by the body to function properly.

Another company also selling glyconutrients is shown here: http://www.naturalcureguide.com/glyconutrients.html Again, as Mannatech does, this company also provides the greatest amounts of the carbohydrates that are not in short supply in the body.

While all of the companies selling glyconutrients have products specifically orientated to correct certain illnesses none of them have anything that addresses the common cold, cold remedies, memory or cell aging. Clearly these missing carbohydrates have capabilities in these areas but these areas are not being addressed by any of the current glyconutrient companies.

One of the short comings with the current offerings to the public is the lack of these missing carbohydrates at therapeutic dose levels (levels at which changes are seen in hours instead of many months). For example, L-Fucose is available in Gum Tragacanth ($34/pound) and Brewer's Yeast (half a pound for $6), but the availability of L-Fucose in Gum Tragacanth is only 0.1% by weight and only 0.05% in Brewer's Yeast. Currently available glyconutrient supplements supply less than 0.1 mg of L-Fucose per daily dose for a 150 pound mammal.

N-Acetyl-Galactosamine is available in shark cartilage (3 oz. $16) but there is only 0.01% by weight of it there. By taking the specially made glyconutrient supplements the daily dose of N-Acetyl-Galactosamine is still generally under 0.1 mg. Likewise, N-Acetyl-Neuraminic Acid is available in Whey Protein (36 26 gram servings for $33) and Hen's eggs but there is only 0.02% of it there by weight. A daily dose from one of the special glyconutrient supplements generally has less than 0.2 mg of it available.

Mannatech even admits that their formulations are extremely weak and require many months to see any results at all “Be patient! Research shows that it may take up to 4 months (or more) to notice the effects of any changes you make to your diet.” https://www.mannatech.com/Shopping/Product.aspx and also see https://www.mannatech.com/Shopping/RDReports.aspx

There have been numerous complaints that Manntech products do not provide any benefit at all even after months of usage; http://www.reviewcentre.com/reviews94020.html “I saw Mannatech Ambrotose, Glyconutrients advertised on a Fibromyalgia site. I was contacted by a Mannatech Associate and was advised that Ambrotose could help with all my ailments—Fibromyalgia, Osteoarthritis, Asthma, allergies etc. I was also told that I wouldn't need to continue to take the vitamin, mineral and herbal supplements I had been using. Now, 4 months on, my asthma has worsened, my cholesterol levels have gone up and my fibromyalgia and arthritis are unchanged.”

From this it can be seen that most of the currently available special glyconutrient supplements sold to replace these missing carbohydrates contain mainly filler material and other chemicals that are already available to the body through other sources or are not needed. Additionally, many of these special supplements contain ingredients known to excite an allergic reaction in a large part of the population; such as Whey Powder and Aloe Vera ingredients. On the other hand there is no known allergic reaction to these essential sugars when taken in their pure form and at therapeutic dose levels.

One of the essential sugars (a carbohydrate), N-Acetyl-Neuraminic Acid (Sialic Acid), has been shown to be more than 1000 times more effective at killing viruses than any other known medication when used in therapeutic doses “Another study reported in a 1995 issue of Antimicrobial Agents and Chemotherapy, stated that a sialic acid mixture was up to 1000 times more effective in fighting influenza than potent antiviral drugs. Such viruses can also cause cold sores, hepatitis, viral pneumonia, as well as the common cold.” http://www.glyconutrients-center.org/N-acetylneuraminic-acid.php

Even though the effectiveness of Sialic Acid as an antiviral agent been known for over a decade it is still waiting to be offered to the public in therapeutic dose levels.

It is therefore an object of the invention to aid the body's ability to fight colds and infections by providing it with the correct mix and level of carbohydrates needed by the body to fend off the infection.

It is another object of the invention to aid the body's ability to lessen the cells aging process by providing it with the correct mix and level of carbohydrates needed by the cells to keep them healthy.

It is another object of the invention to give an animal the ability to fight viruses and infections much more effectively by providing it with the correct mix and level of carbohydrates needed by its body.

It is another object of the invention to promote good health and wellness to all types of mammals through the proper mix of carbohydrates targeted to specific ailments, like increasing mental ability and learning, wound repair and long term memory.

### SUMMARY OF THE INVENTION

In accordance with the present invention, there is to provide a dietary supplement that provides a mammal with the essential carbohydrates needed to maintain proper health and functionality, to fend off illness, lessen the aging process of cells and to provide pets with another level of medications equal to that for humans and specifically to provide an alternative treatment for exercise induced asthma and asthma related illnesses through the use of compounds of the essential carbohydrates.

### BRIEF DESCRIPTION OF THE DRAWINGS

There are no drawings.

### DESCRIPTION OF THE PREFERRED EMBODIMENT

The carbohydrates included in the dietary supplement of the invention are available from a number of manufactures. Most are derived synthetically from other pure chemicals rather than being plant or animal derivatives. A supplier for the two most expensive essential sugars, Sialic Acid (CAS#131-48-6) and N-Acetyl-Galactosamine (CAS#1811-31-0), is R&S PharmChem located in China http://www.rspharmchem.com. A supplier for L-Fucose (CAS#2438-80-4) is AppliChem located in Germany http://www.applichem.de/perl/catalog/catalog.pl. AppliChem can also supply two other more readily available essential sugars, D-Galactose (CAS#59-23-4) and D-Xylose (CAS#58-86-6). D-Mannose (CAS#3458-28-4) is available from a number of the larger supplement suppliers like NOW Foods http://www.nowfoods.com/. The remaining carbohydrate, N-Acetyl-Glucosamine (CAS#98632-70-3) is generally used in one of its many derivative forms. This invention uses the derivatives Glucosamine HCL (CAS#66573-21-5) and Glucosamine Sulfate (CAS#29031-19-4) instead of Glucosamine (CAS#3416-24-8). Both of these carbohydrates are readily available at drug stores. It should be recognized that the composition of the carbohydrate is not intended to be limited by the source from which it is obtained.

It should be stressed that this invention does not incorporate the use of Glucose or Acetylated Mannose. While Glucose is one of the eight essential sugars it is so prevalent in today's diets that adding additional amounts of Glucose in a supplement generally provides no useful benefit. Acetylated Mannose is a plant derivative from the Aloe Vera plant that has not been shown by independent research to be of any beneficial use as a dietary supplement.

Although the present invention includes the above cited seven essential sugars (carbohydrates), it should be noted that other carbohydrates, nutritional compounds or biologically active or inert compounds can be included in the dietary supplement of the invention. Such other ingredients may include spices, flavorings, buffers, gels, binders, filler material, lubrication material, vitamins and or minerals and/or other such compounds that facilitate the formulation or administration of the inventive dietary supplement. These components can be provided separately to a mammal given said dietary supplement.

Many different types of vitamins and minerals can be included in the dietary supplement of the invention. While a few vitamins and minerals of synthetic origin do possess nutritional value, particular embodiments of the dietary supplement herein can contain nutritionally effective amounts of non-toxic vitamins and minerals obtained predominantly from natural sources.

Other compounds, agents and nutrients can also be included in the dietary supplement of the invention, for example: cellulose, calcium carbonate, stearic acid, amino acids, glycine, essential fibers, essential oils, essential botanicals, essential enteric ecology and flora growth promoters, essential fatty acids, and enzymes.

Independent research indicates that these seven essential sugars are not stored in the body. After ingestion the sugars are assimilated into the blood stream within minutes (if taken on an empty stomach). Once in the blood stream they flow through the body and cells in need of these nutrients take what they need and the rest flows on. Most of these unused sugars are excreted via the urine within 12 hours after ingestion. The tests indicate that while excess of these sugars are excreted from the body within 12 hours the cells maintain a internal supply of these sugars for a period of up to a week. Studies have also shown that taking these seven essential sugars, in the levels covered by this invention, did not cause an abnormal rise in the blood sugar levels of diabetics.

The dietary supplement form of the invention has been prepared and can be administered to mammals in powdered, reconstitutable powder, liquid-solid suspension, liquid, capsule and tablet dosage forms. It should be readily obvious to one of ordinary skill in the science of formulations that the present dietary supplement can also be formulated appropriately for irrigation, ophthalmic, rectal, sublingual, transdermal buccal, vaginal, or dermal administration. Thus, other dosage forms such as chewable candy bar, concentrate, drops, elixir, emulsion, film, gel, granule, chewing gum, injection, jelly, oil, paste, pastille, pellet, shampoo, rinse, soap, sponge, suppository, swab, syrup, chewable gelatin form, or chewable tablet can be used.

Due to varying diets among people, the dietary supplement of the invention can be administered in a wide range of dosages and formulated in a wide range of dosage unit strengths. For example, for those people who are missing from their diet seven of the eight essential carbohydrates, a dietary supplement containing those carbohydrates in nutritionally effective amounts can be formulated. As well, for those people whose bioabsorption of essential carbohydrates is extremely efficient, a dietary supplement formulation containing reduced amounts of essential carbohydrates can be prepared.

It should be noted that the dosage of the dietary supplement can also vary according to a particular ailment or disorder that a mammal is suffering from when taking the supplement. For example, a person suffering from chronic colds will generally require a dose different than an animal would who is sick in order to obtain a benefit. An appropriate dose of the dietary supplement can be readily determined by monitoring patient response, i.e., general health, to particular doses of the supplement. As well, when another agent such as a vitamin and/or a herbal extract is being administered to a mammal along with the present carbohydrate dietary supplement, the appropriate doses of the supplement and each of the agents can be readily determined in a like fashion by monitoring patient response, i.e. general health, to particular doses of each.

It is contemplated by the invention that the dietary supplement can be administered simultaneously or sequentially in one or a combination of dosage forms. While it is possible and even likely that the present dietary supplement will provide an immediate overall health benefit, such benefit may take hours or days to materialize. Nonetheless, the present carbohydrate dietary supplement will provide a beneficial nutritional response in a mammal consuming it.

This invention provides an alternative method to treat exercise induced asthma and other asthma related illnesses that is much safer than any of the current treatments. A compound of essential sugars are taken once a week orally by capsule. Lasting results are seen after two months of taking these capsules. As the compound is comprised essentially of sugars, albeit it very special sugars, the compound is not foreign to the body and produces no long term or lasting side effects to most patients.

For the examples herein, the dietary supplement of the invention was administered as a powder-containing capsule. According to the capsule size and ingredients used in a given study exemplified herein, the dietary supplement was administered by oral ingestion. The indicated doses for humans in Example 1 are based upon #00 sized capsules and a #1 size capsule in Examples 2 to 7.

### EXAMPLE 1

A suitable composition for a product according to the present invention is shown in the following table.

| | | | Human |
|---|---|---|---|
| Carbohydrate | Weight % (range) | Weight % (tested) | (mg) |
| D-Galactose | 0.1 to 40 | 6.3 | 43 |
| L-Fucose | 0.1 to 90 | 3.1 | 21 |
| D-Mannose | 10 to 70 | 52.1 | 358 |
| D-Xylose | 0.1 to 70 | 6.3 | 43 |
| Glucosamine HCL | 10 to 60 | 25.1 | 173 |
| Sialic Acid | 0.1 to 50 | 6.5 | 45 |
| N-Acetyl-Galactosamine | 0.1 to 90 | 0.6 | 4 |

In this combination the ingredients Glucosamine HCL, D-Galactose and D-Mannose are optional and preferred. Instead of using Rice Flour as an inactive filler as is done in most products currently available to the public the three optional ingredients set out above were used as active fillers. A disadvantage of using Rice Flour as an inactive filler is its high glycemic index tends to drive a mammal's Triglyceride levels up very high, while using essential sugars as fillers doesn't.

The ingredients are typically in a powered form and are dry blended in a mixer. The mixture can then be packaged as a blended powder into capsules or caplets. In this example the mixture was packaged into size 00 capsules with an average weight of 687 mg for human doses and 25 mg for animal doses. The mg per ingredient for animals would be found by dividing the human ingredient dose in mg by 27.48, for example: for D-Mannose the animal ingredient dose would be 13.0 mg.

This composition is considered a health maintenance mix. It was intended to reduce the probability of infections, like colds, while taking the composition. There were three test trials run using this composition on both humans and animals. These three tests ran from late January 2007 to late December 2007. In the first test, that lasted for two weeks, a capsule/dose was administered twice a day for two weeks. The next test lasted for one month during which time one capsule/dose was administered per day at bedtime.

The third test lasted slightly over nine months and the dose was one capsule/dose a week administered at bedtime. During that time there were no deaths or adverse reactions to the composition by anyone in the test group either human or animal. Regular blood, lipid and electrolyte testing was done. A base line was run prior to the test and during the test blood testing was done regularly to determine if there were any adverse effects due to taking the composition.

The end result was that no one taking this composition contracted a cold or any other type of viral infection during the entire period of the test, even though one of the individuals in the test was prone to chronic colds and flu. This test extended through two different flu seasons and the test subjects continued to work everyday and come in contact with infected people on a daily basis yet they didn't catch anything while taking this composition.

In a follow up test these capsules were taken on a once a month basis by the same human test group during the flu season. Within two weeks following a monthly dose (this was during the four week pause after the end of the weekly tests) two of the subjects came down with a cold or flu. After this the test was stopped and no additional capsules were taken and within two weeks all the test subjects resumed their normal activity of catching colds as normal.

During the animal tests old cats were exposed to new cats infected with FPV on two occasions. Nothing special was done for the first exposure. The second time an additional dose was given just after exposure. After these two exposures, which were several months apart, all of the old cats tested positive for FPV. However, none of the cats came down with any symptoms and are in excellent health at the time of this writing.

### EXAMPLE 2

In a different embodiment of this invention another suitable composition for a product according to the present invention is shown in the following table:

| | | | Human |
|---|---|---|---|
| Carbohydrate | Weight % (range) | Weight % (tested) | (mg) |
| D-Galactose | 0.1 to 50 | 7.2 | 25 |
| L-Fucose | 0.1 to 90 | 14.4 | 50 |
| D-Mannose | 1.0 to 70 | 31.6 | 110 |
| D-Xylose | 0.1 to 70 | 10.8 | 38 |
| Glucosamine HCL | 1.0 to 50 | 10.8 | 38 |
| Sialic Acid | 0.1 to 50 | 10.8 | 38 |
| N-Acetyl-Galactosamine | 0.1 to 90 | 14.4 | 50 |

In this combination the ingredients Glucosamine HCL and D-Mannose are optional and preferred. In this composition D-Galactose is not considered to be optional as this composition is intended for animals as well as humans.

This composition is considered a wellness mixture. It was intended to reduce the probability of infections, like colds, while taking the capsules. These ingredients provide Anti-Viral, Anti-Fungal and defense against Gram Negative Bacteria. This combination was initially derived from the findings of independent researchers. In recent studies in 2008 this composition was also found to be extremely effective at mitigating the effects of handover due to over consumption of alcohol.

### EXAMPLE 3

In a different embodiment of this invention another suitable composition for a product according to the present invention is shown in the following table:

| | | | Human |
|---|---|---|---|
| Carbohydrate | Weight % (range) | Weight % (tested) | (mg) |
| D-Mannose | 1.0 to 40 | 28.8 | 100 |
| Glucosamine Sulfate | 0.1 to 30 | 20.8 | 72 |
| D-Xylose | 0.1 to 70 | 28.8 | 100 |
| Sialic Acid | 0.1 to 50 | 21.6 | 75 |

In this combination the ingredient D-Mannose is optional and preferred. This composition was packaged in a size #1 capsule and taken twice a day. This combination is good for both humans and animals.

This composition is considered a cold pill mix. It is intended to reduce the effects of viral infections, like colds, and speed recovery. These ingredients provide Anti-Viral, Anti-Fungal and defense against Gram Negative Bacteria the same as in the wellness mixture, shown in Example 2, except here the amounts have been raised to achieve the maximum beneficial effect.

When taken after the onset of a cold dramatic relief is felt within four hours of taking this composition. The two doses should be taken 12 hours apart and not within two hours of a meal. Generally 10 PM and 10 AM seemed to work best for the test subjects. In some cases just taking a single dose at bedtime was sufficient to completely end all of the symptoms by morning.

### EXAMPLE 4

In a different embodiment of this invention another suitable composition for a product according to the present invention is shown in the following table:

| | | | Human |
|---|---|---|---|
| Carbohydrate | Weight % (range) | Weight % (tested) | (mg) |
| L-Fucose | 0.1 to 80 | 21.6 | 75 |
| Glucosamine HCL | 1.0 to 50 | 28.0 | 97 |
| Sialic Acid | 0.1 to 50 | 21.6 | 75 |
| N-Acetyl-Galactosamine | 0.1 to 90 | 28.8 | 100 |

In this combination the ingredient Glucosamine HCL is optional and preferred. This composition was packaged in a size #1 capsule and taken once a day. It is good for both humans and animals.

This composition is considered an anti-aging plus learning and memory enhancer mixture. It is intended to reduce the effects of cell aging and enhance memory and learning ability. This combination will not reverse any current level effects already present but should help to reduce the rate at which the cells age progressively. This combination was derived from the findings of independent researchers. Most of the research relating to aging using the essential sugars was done with animals.

### EXAMPLE 5

In a different embodiment of this invention another suitable composition for a product according to the present invention is shown in the following table:

| | | | Human |
|---|---|---|---|
| Carbohydrate | Weight % (range) | Weight % (tested) | (mg) |
| L-Fucose | 0.1 to 90 | 28.8 | 100 |
| Glucosamine HCL | 1.0 to 50 | 42.4 | 147 |
| Sialic Acid | 0.1 to 60 | 28.8 | 100 |
| N-Acetyl-Galactosamine | 0.0 to 1 | 0.0 | 0 |

In this combination the ingredient Glucosamine HCL is optional and preferred. This composition was packaged in a size #1 capsule and taken once a day. It is good for both humans and animals. While this composition uses the same ingredients as Example 4 the amount of N-Acetyl-Galactosamine has been reduced to zero for this use.

This composition is considered a learning enhancer mixture. It is intended to increase the ability of one to learn new tasks and to improve memory. This combination was derived from the findings of independent researchers. Most of the research relating to learning ability using the essential sugars was done with animals.

### EXAMPLE 6

In a different embodiment of this invention another suitable composition for a product according to the present invention is shown in the following table:

| | | | Human |
|---|---|---|---|
| Carbohydrate | Weight % (range) | Weight % (tested) | (mg) |
| L-Fucose | 0.1 to 90 | 28.2 | 100 |
| Glucosamine Sulfate | 0.1 to 25 | 22.5 | 80 |
| Sialic Acid | 0.1 to 80 | 14.1 | 50 |
| N-Acetyl-Galactosamine | 0.1 to 90 | 35.2 | 125 |

In this combination the ingredient Glucosamine Sulfate is optional and preferred. This composition is packaged in a size #1 capsule and is taken once a week. It is good for both humans and animals in helping to relieve breathing difficulties due to asthma related illnesses.

### EXAMPLE 7

In a different embodiment of this invention another suitable composition for a product according to the present invention is shown in the following table:

| | | | Human |
|---|---|---|---|
| Carbohydrate | Weight % (range) | Weight % (tested) | (mg) |
| L-Fucose | 0.1 to 90 | 34.7 | 125 |
| Glucosamine HCL | 0.1 to 25 | 23.6 | 85 |
| N-Acetyl-Galactosamine | 0.1 to 95 | 41.7 | 150 |

In this combination the ingredient Glucosamine HCL is optional and preferred. This composition is packaged in a size #1 capsule and is taken once a week. It is good for both humans and animals. While other essential sugars/carbohydrates could have been added to this composition, such as Mannose, Galactose, and Xylose, to enhance the composition the ingredients shown in Example 7 are the least known to be therapeutically beneficial for the treatment of exercise induced asthma and other asthma related diseases as found from the 2008 tests.

This composition was found to be extremely effective at reducing the effects of exercise induced asthma during human tests conducted from March 2008 to June 2008. This carbohydrate combination needs to be taken once a week, at bedtime on an empty stomach, for two months to gain the maximum benefits from the composition. This carbohydrate combination was derived and refined from the results of the 2008 tests. After taking the composition for two months strenuous outdoor exercise was conducted in elevated temperatures of 90 degrees F. which including traversing a 600′ elevated grade over a half mile course with a 30 minute time limit to complete the course.

One of the subjects suffered from extreme GERD and had been a chronic exercise induced asthma patient for a number of years. Similar exercise in the past had almost incapacitated him due to the heavy coughing and wheezing for air. However, after taking the composition once a week for just two months he no longer needed an inhaler after such exercise and wasn't bothered by any of the coughing or wheezing episodes that use to routinely accompany strenuous exercise. The other members of the test group were not as bad an asthma sufferer as this subject but they all experienced similar recuperating effects including the loss of the associated wheezing and coughing that had always been associated with exercise.

The novelty of the present invention is that it has no known side effects and it only needs to be administered once a week by taking one small capsule and the effects last all week. This represents a major advantage over the daily and hourly medications and inhalers presently being used to treat EIA. All of the current EIA medications have been shown to exhibit undesirable side effects and some exhibited extremely serious life threatening side effects. As the present invention uses carbohydrate compounds that don't have any known side effects to treat EIA this also represents a significant improvement over all present medications as far as health and safety considerations are concerned.

It can be seen that this invention has a large number of possible beneficial compositions utilizing just one, or any combination, of the seven essential sugars specialized for a specific target. Just because a combination is not specifically set out herein should not limit the scope of this invention. It has been sufficiently shown that there are numerous compositions available with useful purposes by the detailed examples set out herein.

Additionally, the weighting of a composition if varied by ingredient will specify the composition for a new target use even though the ingredients for two different target uses are the very same, refer to Examples 4 and 5. By changing the amounts of an ingredient its effects on cell absorption will change and by increasing or reducing an ingredient within a cell it will turn on or off different genes which will alter the body's response; The Geno Type Diet by Dr. Peter J. D'Adamo, Broadway Books, 2007, ISBN 978-0-7679-2524-2.

In summary, this invention pertains to the field of dietary supplements and nutritional support for promotion and maintenance of optimal good health. More specifically, the invention relates to compositions of seven essential sugars/carbohydrates as dietary supplements that are essential for a mammal's optimal health and functionality.

This invention will correct the problem caused by modern diets consisting of highly refined foods, from which many essential ingredients have been eliminated during processing, specifically the seven essential sugars needed for a properly functioning mammal. It will also cure the problem inherent in most of the glyconutrients available today that contain only trace amounts of these essential sugars while containing large amounts of inactive ingredients that can and do cause numerous allergic reactions with no benefit realized.

The above is a detailed description of particular embodiments of the invention. Those of skill in the art should, in light of the present disclosure, appreciate that obvious modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

Since other modifications and changes varied to fit particular operating requirements and environments will be apparent to those skilled in the art, the invention is not considered limited to the examples chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A composition of at least three or more carbohydrate(s) selected from the following group: galactose, mannose, n-acetylneuraminic acid, fucose, n-acetylgalactosamine, xylose, glucosamine, glucosamine HCL, glucosamine sulfate;
wherein said composition is used as a beneficial treatment for asthma related illnesses.

2. The compositions of carbohydrates in accordance with claim 1, wherein said composition further comprises a flowing agent and or a lubricant.

3. The compositions of carbohydrates in accordance with claim 1, further comprising one or more non-toxic vitamins and or minerals.

4. The compositions of carbohydrates in accordance with claim 1, wherein said composition further comprises a filler ingredient(s).

5. The compositions of carbohydrates in accordance with claim 1, further comprising one or more non-toxic herbal, fungal, plant and or animal derived agents.

6. The compositions of carbohydrates in accordance with claim 1, administered to humans or animals using any of the following methods: capsule, caplet, tablet, liquid, suppository, drops, paste, injection, pellet, chewable.
